(19) United States
(12) Patent Application Publication
(10) Pub. No.: US 2005/0107306 A1
(43) Pub. Date: May 19, 2005

(54) Treatment of respiratory disease associated with matrix metalloproteases by inhalation of synthetic matrix metalloprotease inhibitors

(76) Inventors: Philip J. Barr, Oakland, CA (US); Philip A. Pemberton, San Mateo, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/848,594

(22) Filed: May 17, 2004

Related U.S. Application Data
(60) Provisional application No. 60/471,283, filed on May 16, 2003.
(60) Provisional application No. 60/471,896, filed on May 19, 2003.
(60) Provisional application No. 60/517,937, filed on Nov. 5, 2003.

(51) Int. Cl. A61K 38/04

(52) U.S. Cl. 514/19

(57) ABSTRACT

The present invention encompasses methods and compositions for the treatment and prevention of respiratory diseases associated with matrix metalloproteases. More specifically, the present invention relates to the treatment and prevention of respiratory diseases associated with matrix metalloproteases by inhalation of synthetic matrix metalloprotease inhibitors. Exemplary respiratory diseases associated with matrix metalloproteases that may be treated by the methods of the invention include chronic obstructive pulmonary disease, emphysema, asthma, cystic fibrosis, and chronic bronchitis. An exemplary synthetic matrix metalloprotease useful in the methods of the invention is ilomastat.

### CROSS-REFERENCE TO RELATED APPLICATIONS

This application claims priority to U.S. Provisional Patent Application No. 60/471,283, filed May 16, 2003, U.S. Provisional Patent Application No. 60/471,896, filed May 19, 2003, and U.S. Provisional Patent Application No. 60/517,937, filed Nov. 5, 2003, all entitled “Treatment of Respiratory Disease Associated with Matrix Metalloproteases by Inhalation of Synthetic Matrix Metalloprotease Inhibitors.” These applications are incorporated herein by reference in their entirety.

### FIELD OF THE INVENTION

The present invention relates to methods and compositions for the treatment and prevention of respiratory diseases associated with matrix metalloproteases. More specifically, the present invention relates to the treatment and prevention of respiratory diseases associated with matrix metalloproteases by inhalation of synthetic matrix metalloprotease inhibitors.

### BACKGROUND OF THE INVENTION

Respiratory diseases associated with an imbalance between pulmonary matrix metalloproteases and pulmonary matrix metalloprotease inhibitors, such as chronic obstructive pulmonary disease (COPD), emphysema, asthma, cystic fibrosis, and chronic bronchitis, are a major cause of morbidity and mortality worldwide. There have been various descriptions of MMPs, MMPIs, and/or MMP-related diseases; see, e.g., PCT Application Nos. WO 98/52575, WO 01/22952, WO02089730A2, WO02089730A2, WO0038718A2, U.S. Pat. Nos. 4,666,897; 5,114,953; 5,183,900; 5,773,430; 5,773,438; 5,872,152; 5,892,112; 6,417,229; 6,352,976; 6,197,770; 6,121,258; 6,465,468; 6,483,193; 6,608,112; and Brown (2000) Exp. Opin. Invest. Drugs 9:2167-2177; Giavazzi et al. (1998) Clin. Cancer Res. 4)985-92; Shalinsky et al. (1999) Clin. Cancer Res. 5:1905-17; Shalinsky et al. (1998) Invest. New Drugs 16:303-13; Erlichmann et al. (2001) Ann. Oncol. 12: 389-95; Bramhall et al. (2001) J. Clin. Oncol. 19:3447-55; Martin et al. (2001) Prog Respir. Res. 31: 177-180; and Ilomastat Fact Sheet (CalBiochem). However, a need still remains for an effective method of treating and/or preventing these diseases.

### SUMMARY OF THE INVENTION

In one aspect, the invention encompasses methods of treating an individual who suffers from a respiratory disease associated with matrix metalloproteases by administering to the individual an effective amount of a synthetic matrix metalloprotease inhibitor by inhalation. In another aspect, the invention encompasses methods of preventing a respiratory disease associated with matrix metalloproteases in an individual who is susceptible to or who suffers from a respiratory disease associated with matrix metalloproteases by administering to the individual an effective amount of a synthetic matrix metalloprotease inhibitor by inhalation, wherein preventing the disease encompasses eliminating the appearance, increasing the time to appearance, delaying or slowing the development, and/or decreasing the number and severity of clinical and other manifestations.

In some embodiments, the individual is a mammal. In some embodiments, the individual is a human. In some embodiments, the human is a smoker.

In some embodiments of the invention, the respiratory disease is chronic obstructive pulmonary disease (COPD). In other embodiments, the respiratory disease is emphysema, either hereditary or environmental. In some embodiments, the respiratory disease is environmental emphysema (and in some embodiments, the individual is a smoker). In some embodiments the respiratory disease is hereditary emphysema. In further embodiments, the respiratory disease is asthma. In yet further embodiments, the respiratory disease is cystic fibrosis. In still yet further embodiments, the respiratory disease is chronic bronchitis.

In some embodiments of the invention, the matrix metalloprotease inhibitor is a hydroxamate-based synthetic matrix metalloprotease inhibitor. In some of these embodiments, the hydroxamate-based synthetic matrix metalloprotease inhibitor comprises a binding structure that includes an isobutyl and/or tryptophan moiety. In some embodiments, the synthetic MMPI is selected from the group consisting of ilomastat, RS-113456, and RS-132908. In some embodiments, the synthetic matrix metalloprotease inhibitor is ilomastat. In some of the latter embodiments, the ilomastat is administered in a daily dose (in some embodiments, as a single dose and in some embodiments as divided doses) of about 1 mg to about 10 mg. In some of these embodiments, the ilomastat is administered in a daily dose (in some embodiments, as a single dose and in some embodiments as divided doses) of about 1 mg. In some of these embodiments, the ilomastat is adminstered in a cumulative daily dose of about 0.1 mg to about 1 mg.

In some embodiments of the invention, the synthetic matrix metalloprotease inhibitor is administered in a daily dose (in some embodiments, as a single dose and in some embodiments as divided doses) from a lower limit of any of about 0.05, 0.1, 0.5, 1, 2, 3, 4, 6, 8, 10, 15, 20, 30, 40 or 50 mg to an upper limit of any of about 0.1, 0.5, 1, 2, 4, 6, 8, 10, 15, 20, 30, 40, 50, or 100 mg.

In some embodiments, other active agents are administered in combination with the synthetic matrix metalloprotease inhibitor. In some of these embodiments, the other active agent is a bronchodilator and/or a corticosteroid. In some embodiments, an MMPI is delivered in combination with a bronchodilator once daily. In some embodiments, an MMPI is delivered in combination with a bronchodilator twice daily. In some embodiments, ilomastat is delivered in combination with a bronchodilator once daily. In some embodiments, ilomastat is delivered in combination with a bronchodilator twice daily. In some embodiments, the bronchodilator is oxitropium bromide, ipratropium bromide, or tiotropium bromide.

In some embodiments, the methods of the invention encompass treating an individual who suffers from a respiratory disease associated with matrix metalloproteases, or preventing a respiratory disease associated with matrix metalloproteases in an individual who is susceptible to or who suffers from a respiratory disease associated with matrix metalloproteases, by administering to the individual an effective amount of ilomastat by inhalation. In some embodiments, the daily dose (in some embodiments, as a single dose and in some embodiments as divided doses) of ilomastat is from about 1 mg to about 10 mg. In some embodiments, the daily dose (in some embodiments, as a single dose and in some embodiments as divided doses) of ilomastat is about 1 mg. In some embodiments, the disease is COPD, emphysema (hereditary or environmental), asthma, cystic fibrosis, or chronic bronchitis. In some embodiments, the disease is emphysema. In some embodiments, the disease is asthma. In some embodiments, the individual is a smoker. In some embodiments, the individual is a passive smoker. In some embodiments, the individual is an active smoker.

In some embodiments, the invention encompasses a method of treating emphysema in an individual (in some embodiments, in a smoker) by administering to the individual (in some embodiments, a smoker) a daily dose (in some embodiments, as a single dose and in some embodiments as divided doses) of ilomastat of from about 1 mg to about 10 mg by inhalation. In some of these embodiments, the individual is a passive smoker; in other embodiments, the individual is an active smoker. In some embodiments, the daily dose (in some embodiments, as a single dose and in some embodiments as divided doses) of ilomastat is about 6 mg. In some embodiments, the daily dose (in some embodiments, as a single dose and in some embodiments as divided doses) of ilomastat is about 1 mg. In some embodiments, the cumulative daily dose is about 0.1 mg to about 1 mg.

In some embodiments, the invention encompasses a method of preventing emphysema in an individual (in some embodiments, a smoker) by administering to the individual (in some embodiments, a smoker) a daily dose (in some embodiments, as a single dose and in some embodiments as divided doses) of ilomastat of from about 1 mg to about 10 mg by inhalation, wherein preventing emphysema encompasses eliminating the appearance, increasing the time to appearance, delaying or slowing the development, and/or decreasing the number and severity of clinical and other manifestations of emphysema. In one embodiment, the daily dose (in some embodiments, as a single dose and in some embodiments as divided doses) of ilomastat is about 6 mg. In one embodiment, the daily dose (in some embodiments, as a single dose and in some embodiments as divided doses) of ilomastat is about 1 mg. In some embodiments, the cumulative daily dose is about 0.1 mg to about 1.0 mg.

In some embodiments, the invention encompasses a method of treating or preventing hereditary emphysema in an individual who is susceptible to hereditary emphysema by administering to the individual an MMPI by inhalation. In some of these embodiments, the MMPI is ilomastat and the daily dose (in some embodiments, as a single dose and in some embodiments as divided doses) is from about 1 mg to about 10 mg.

In some embodiments, the invention encompasses treating asthma in an individual by administering to the individual an effective amount of an MMPI by inhalation. In some of these embodiments, the MMPI is administered at a dose of about 0.1 mg to about 1 mg per day; in some embodiments, the MMPI dose is about 0.3 mg per day. In some of these embodiments, the MMPI is ilomastat. In some of these embodiments, the individual is a human. In one embodiment, the invention provides a method for treating asthma in a human who suffers from asthma by administration of about 0.3 mg per day of ilomastat to the human by inhalation.

### BRIEF DESCRIPTION OF THE FIGURE

FIGURE 1 is a representation of the chemical structure of ilomastat.

### DETAILED DESCRIPTION OF THE INVENTION

The invention encompasses methods, compositions, and kits for preventing, treating, inhibiting, or delaying the development of respiratory disease associated with matrix metalloproteases through inhalation of an effective amount of a synthetic matrix metalloprotease inhibitor. In some embodiments, the methods of the invention are directed at treating an existing respiratory disease associated with matrix metalloproteases. In some embodiments, the methods of the invention are directed at preventing, e.g., eliminating the appearance, increasing the time to appearance, delaying or slowing the development, and/or decreasing the number and severity of clinical and other manifestations, of a respiratory disease associated with matrix metalloproteases. In some embodiments, the respiratory disease associated with matrix metalloproteases is COPD; in some embodiments, the respiratory disease associated with matrix metalloproteases is emphysema; in some embodiments, the respiratory disease associated with matrix metalloproteases is asthma; in some embodiments, the respiratory disease associated with matrix metalloproteases is cystic fibrosis; in some embodiments, the respiratory disease associated with matrix metalloproteases is chronic bronchitis. It will be appreciated that the methods of the invention may be useful in more than one of the above contexts simultaneously; e.g., in treating an individual suffering from one form of a respiratory disease associated with matrix metalloproteases; while also delaying or preventing the onset of another form of a respiratory disease associated with matrix metalloproteases.

The methods of the invention involve inhalation of an effective amount of a synthetic matrix metalloprotease inhibitor (MMPI) by an individual suffering from or susceptible to a respiratory disease associated with matrix metalloproteases. In some embodiments, other active ingredients are administered in combination with the MMPI. Formulations and compositions of the invention include suitable excipients and other ingredients for inhalation, as are known in the art. Kits of the invention may include compositions of the invention, in suitable packaging, together with other components such as instructions, administration devices, and diluents.

In some embodiments of the invention the synthetic MMPI used is one wherein the zinc-chelating activity resides in a hydroxamate moiety. In some embodiments, the synthetic MMPI may be chosen from a hydroxamate-based MMPIs in the group consisting of ilomastat, batimastat, marimastat, prinomastat, CGS 27023A, BAY 12-9566, Ro 32-3555, RS-113456, RS-132908, and RS-130,830. In some embodiments, the synthetic MMPI is selected from the group consisting of ilomastat, RS-113456, and RS-132908. In some embodiments, the synthetic MMPI may be chosen from a hydroxamate-based MMPIs in the group consisting of ilomastat and related compounds disclosed in Levy et al. (1998) J. Med. Chem. 41:199-223, and Table 1 of U.S. Pat. No. 5,183,900.

In some embodiments of the invention, the synthetic MMPI used is ilomastat. Strikingly, ilomastat has been found to reduce or eliminate indices of smoking-induced COPD (e.g., emphysema) at low doses. See Example 2. The hydroxamate-containing MMPIs RS-113456, and RS-132908 have been shown to be effective against smoking-induced COPD when delivered orally, but at oral doses several hundred-fold greater than those used for pulmonary administration in the present invention. See, e.g., Brown (2000) Exp. Opin. Invest. Drugs 9: 2167-2177; Martin et al. (2001) Prog Respir. Res. 31: 177-180. In some embodiments, other active agents that ameliorate one or more symptoms of COPD may be used in combination with a synthetic MMPI. These agents include, but are not limited to, bronchodilators, and corticosteroids.

The methods of the invention encompass inhalation of a synthetic MMPI. Formulations of the invention include those that are suitable for storage of the synthetic MMPI and, if included, other active agents, and for administration by inhalation. In some embodiments the methods and compositions (formulations) of the invention encompass dry powder formulations and/or their inhalation. In other embodiments, the method and compositions of the invention encompass liquid formulations and/or their inhalation. Methods of administration by inhalation include inhalation from a nebulizer, inhalation from a dry powder inhaler, and inhalation from a metered dose inhaler. The synthetic MMPI and/or other active agents are administered at a dosage, frequency, and duration sufficient to prevent, treat, inhibit, and/or delay the development a respiratory disease associated with MMPs in the individual to which the synthetic MMPI is being administered. Treatment efficacy is assessed by assessment of the reduction of, or halting or slowing of progression of, clinical manifestations of COPD.

In one embodiment, the invention encompasses inhalation of a synthetic MMPI by a smoker to prevent or treat smoking-induced respiratory disease associated with MMPs. In some of these embodiments the synthetic MMPI is ilomastat. In some of these embodiments, the smoking-induced respiratory disease associated with MMPs is emphysema. In some embodiments, the smoker is an active smoker, e.g., one who currently smokes tobacco.

Compositions of the invention contain at least one synthetic MMPI. In one embodiment, the MMPI is ilomastat. Compositions may further include other active agents, such as bronchodilators and/or steroids. Optionally, compositions of the invention may further include excipients, stabilizers, preservatives and the like.

Kits of the invention may include the compositions of the invention, in suitable containers, and any materials necessary or useful in the administration and use of the compositions in the treatment and/or prevention of a respiratory disease associated with matrix metalloproteases, such as COPD.

General Techniques

The practice of the present invention will employ, unless otherwise indicated, conventional techniques of medicine and pharmacology, which are within the skill of the art. Such techniques are explained fully in the literature, such as Wyngaarden, et al. (eds.), Textbook of Medicine, 19^{th} ed., W.B. Saunders Co., Philadelphia, 1992; Gillman et al. (eds.) (1990) Goodman and Gilman's: The Pharmacological Bases of Therapeutics, 8^{th} Ed., Pergaman Press; Norris (ed.) (1989) Novel Drug Delivery Systems, 2^{nd} Ed., Marcell Dekker Inc.; and Remington's Pharmaceutical Sciences, 19^{th} ed. (2000) Mack Publishing Co.

Definitions

As used herein, “treatment” or “treating” is an approach for obtaining beneficial or desired clinical results such as those listed for “effective amount.”

As used herein, “prevention” or “preventing” includes eliminating the appearance, increasing the time to appearance, delaying or slowing the development, and/or decreasing the number and severity of clinical and other manifestations of a respiratory disease associated with matrix metalloproteases that does appear, such as those listed for “effective amount.”

A “respiratory disease associated with matrix metalloproteases” is a disease or pathological condition of the lungs and/or associated structures that is associated with an imbalance between pulmonary matrix metalloproteases (MMPs) and pulmonary matrix metalloprotease inhibitors (MMPIs). In some cases the imbalance occurs because of an excess of one or more MMPs; in some cases the imbalance occurs because of a deficiency of one or more MMPIs; in some cases the imbalance occurs because of both of the preceding. The imbalance leads to greater than normal activity of one or more pulmonary MMPs. In some embodiments of the invention the respiratory disease is COPD; in other embodiments, the respiratory disease is emphysema; in further embodiments, the respiratory disease is asthma; in still further embodiments, the respiratory disease is cystic fibrosis; in yet still further embodiments, the respiratory disease is chronic bronchitis. Clinical manifestations, pathologic findings, standard therapy requirements, and prognoses of COPD, emphysema, asthma, cystic fibrosis, and chronic bronchitis are well-known in the art. See, e.g., National Institutes of Health (2001) Global Initiative for Chronic Obstructive Lung Disease (“The GOLD report”), NIH Publication No. 2701; Wyngaarden, et al. (eds.), Textbook of Medicine, Nineteenth Edition, W. B. Saunders Co., Philadelphia, 1992.

An “individual” is a vertebrate, preferably a mammal, more preferably a human. Mammals include, but are not limited to, farm animals, sport animals, pets, primates, horses, dogs, cats, mice and rats.

An individual “suffers” from a respiratory disease associated with matrix metalloproteases if the individual shows clinical signs (i.e., one or more symptoms, as one skilled in the art would understand) of a respiratory disease associated with matrix metalloproteases; such clinical signs are well-known in the art. See, e.g., National Institutes of Health (2001) Global Initiative for Chronic Obstructive Lung Disease (“The GOLD report”), NIH Publication No. 2701; Wyngaarden, et al. (eds.), Textbook of Medicine, Nineteenth Edition, W.B. Saunders Co., Philadelphia, 1992.

An individual is “susceptible” to a respiratory disease associated with matrix metalloproteases if the individual is known or suspected of having one or more risk factors for a respiratory disease associated with matrix metalloproteases, including a hereditary predisposition to a respiratory disease associated with matrix metalloproteases, exposure to environmental conditions that increase the likelihood of a respiratory disease associated with matrix metalloproteases, and/or any other risk factors for the development of a respiratory disease associated with matrix metalloproteases, compared to an individual without such risk factors. Other risk factors can include, but are not limited to, family history of one or more respiratory disease associated with matrix metalloproteases, history of previous disease, occupation, age, sex, race, diet, or presence of precursor disease. An individual who is exposed to tobacco smoke is susceptible to a respiratory disease associated with matrix metalloproteases, especially to emphysema.

A “smoker,” as used herein, is an individual who is or has been exposed to tobacco smoke, whether occasionally or frequently, and whether as the result of direct use of tobacco products, or as the result of inhalation of secondhand tobacco smoke. Thus the term “smoker” encompasses present and past smokers (i.e., individuals who have quit smoking) and individuals who are or have been exposed to secondhand tobacco smoke in the present or the past.

An “effective amount” of drug, compound, or pharmaceutical composition is an amount sufficient to effect beneficial or desired results including prevention, delay, halting or slowing of progression, or modulation, including amelioration, of one or more clinical manifestations or one or more symptoms of a respiratory disease associated with matrix metalloproteases. Depending on the disease, such clinical manifestations may include dyspnea, coughing, wheezing (especially on forced exhalation), reduction in forced expiratory volume (FEV), reduced arterial PO₂, dependent edema, and recurrent respiratory infection. An “effective amount” for the prevention of a respiratory disease associated with matrix metalloproteases, e.g., in an individual exposed to environmental conditions that increase the likelihood of a respiratory disease associated with matrix metalloproteases, e.g., a smoker, include an amount that prevents the appearance of a respiratory disease associated with matrix metalloproteases, or a delay in the time to appearance of a respiratory disease associated with matrix metalloproteases compared to the expected time to appearance for a similar, untreated individual, or a slowing or halting of a respiratory disease associated with matrix metalloproteases if present or if it does appear, or decreased relapse rates in managed respiratory disease associated with matrix metalloproteases. An “effective amount” can also be an amount that improves quality of life measures, such as physical functioning, bodily pain, general health, vitality, social functioning, decreasing the dose of other medications, e.g. palliative care medications or other medications, required to treat the disease, delaying the progression of the disease, decreasing time required for resolution of secondary infection and/or symptoms, and/or prolonging survival of patients. An effective amount can be administered in one or more administrations. For purposes of this invention, an effective amount of drug, compound, or pharmaceutical composition may be an amount sufficient to decrease clinical manifestations of a respiratory disease associated with matrix metalloproteases.

A “matrix metalloprotease inhibitor (MMPI)” is any substance that reduces or eliminates the activity of one or more matrix metalloproteases (MMPs). A “synthetic MMPI,” as used herein, is a non-naturally occurring substance that reduces or eliminates the activity of one or more MMPs. A synthetic MMPI may have any structure or design, and may reduce or eliminate MMP activity through any mechanism. Synthetic MMPIs include, but are not limited to, those produced by standard organic synthetic methods (e.g., ilomastat and related compounds), and those produced by recombinant methods (e.g., fusion proteins that encompass all or a portion of a polypeptide MMPI).

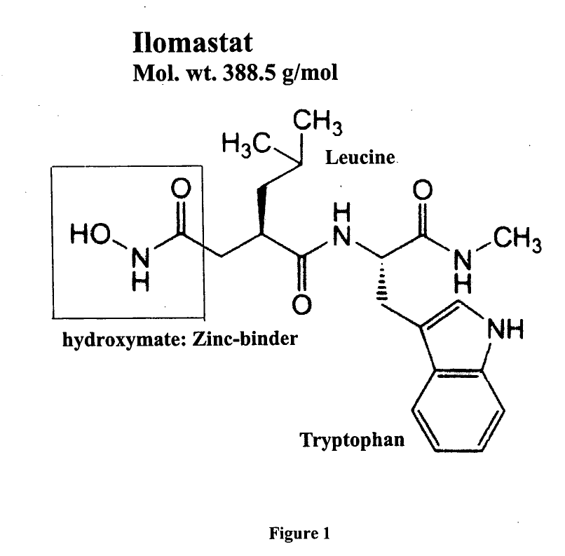

A “hydroxamate-based MMPI” is an MMPI that includes a hydroxamate (see FIG. 1 for structure), and where the hydroxamate chelates the zinc of an MMP, thus inhibiting the MMP. Hydroxamate MMPIs may include various moieties that make them specific for binding pockets of different MMPs, thus determining the specificity of the MMPI. One class of hydroxamate-based MMPIs used in the invention are those that include an isobutyl and/or a tryptophan moiety. Such MMPIs are useful in some embodiments of the invention, and are described in Levy et al. (1998) J. Med. Chem. 41:199-223, and in Table 1 of U.S. Pat. No. 5,183,900, which are hereby incorporated by reference in their entirety.

“Inhalation” refers to a method of administration of a compound that delivers an effective amount of the compound so administered to the tissues of the lower respiratory tract by inhalation of the subject compound by the individual being treated, thereby drawing the compound into the deep lung.

As used herein, the singular form “a”, “an”, and “the” includes plural references unless indicated otherwise. For example, “a symptom” means one or more symptoms.

Methods of the Invention

With respect to all methods described herein, reference to matrix metalloprotease inhibitors (MMPIs) also includes compositions comprising one or more of these agents. These compositions may further comprise suitable excipients, such as pharmaceutically acceptable excipients including buffers, which are well known in the art. The present invention can be used alone or in combination with other conventional methods of treatment.

Respiratory Disease Associated with Matrix Metalloproteases

The invention encompasses methods for treating and/or preventing respiratory disease associated with matrix metalloproteases. In some embodiments of the invention the respiratory disease is COPD; in other embodiments, the respiratory disease is emphysema; in further embodiments, the respiratory disease is asthma; in still further embodiments, the respiratory disease is cystic fibrosis; in yet still further embodiments, the respiratory disease is chronic bronchitis. The methods of the invention are useful both in individuals known to suffer from a respiratory disease associated with matrix metalloproteases, and in individuals who are susceptible to a respiratory disease associated with matrix metalloproteases; the latter includes individuals who are subject to environmental factors and/or genetic conditions that predispose them to a respiratory disease associated with matrix metalloproteases. Non-limiting examples of the environmental factors known to increase the likelihood of a respiratory disease associated with matrix metalloproteases or accelerate the appearance or progress of a respiratory disease associated with matrix metalloproteases are smoking, both firsthand (i.e., active) and secondhand (i.e., passive), exposure to air pollution, and exposure to industrial conditions that increase susceptibility to respiratory disease, such as exposure to cadmium. See, e.g., Leduc et al. (1993) Thorax 48:570-571; Levy et al. (1977) Am. Rev. Resp. Dis. 116:167-173; Hautamaki et al. (1997) Science 277:2002-2004. Stockley (2002) Chest 121:151S-155S. A “smoker,” as used herein, is an individual (human) who is or has been exposed to tobacco smoke, either occasionally or frequently, and either as the result of direct use of tobacco products, or as the result of inhalation of secondhand tobacco smoke. In some embodiments, the methods are used to prevent or treat a respiratory disease associated with matrix metalloproteases in a direct user of tobacco products; in some embodiments, the diseases is COPD; in some embodiments, the disease is emphysema. Non-limiting examples of genetic factors known to increase the likelihood of a respiratory disease associated with matrix metalloproteases include hereditary alpha one-antitrypsin (AAT) deficiency (either heterozygous or homozygous). See, e.g., Hill et al. (2000) Thorax 55:970-977.

In some embodiments, the invention encompasses methods to treat and/or prevent emphysema, which may be of either hereditary or environmental origin (or both). In one embodiment, the invention encompasses methods to treat and/or prevent hereditary emphysema in an individual at risk of developing hereditary emphysema by inhalation of a matrix metalloprotease inhibitor. In one embodiment, the matrix metalloprotease inhibitor is ilomastat. In one embodiment, the invention encompasses methods to treat and/or prevent emphysema in a smoker by inhalation of a matrix metalloprotease inhibitor. In one embodiment, the invention encompasses methods to treat and/or prevent emphysema in a smoker (e.g., a direct, active smoker) by inhalation of ilomastat. It will be appreciated that in some individuals hereditary and environmental factors combine to produce a susceptibility to emphysema that may be treated by the methods of the invention.

In one embodiment, the invention encompasses methods to treat and/or prevent asthma by inhalation of a matrix metalloprotease inhibitor. In one embodiment, the invention encompasses methods to treat and/or prevent asthma by inhalation of ilomastat.

The methods of the invention involve administration of an effective amount of a synthetic matrix metalloprotease inhibitor (MMPI) to the individual to be treated. In some embodiments, other active agents are administered in combination with the MMPI. Formulations of the invention may also include suitable excipients and other ingredients for pulmonary administration, as are known in the art.

Matrix Metalloprotease Inhibitors

Matrix metalloproteases (MMPs) are one class of metalloproteases, and have been found to be particularly important in a number of normal and pathological conditions. They are especially implicated in the development of respiratory diseases associated with matrix metalloproteases. See, e.g., Parks and Shapiro (2001) Respir. Res. 2:10-19; Segura-Valdez et al. (2000) Chest 117:684-694; Shapiro (1999) Am. J. Respir. Crit. Care Med. 160:S29-S32; Tetley (2002) Chest 121:156S-159S; Kelly and Jarjour (2003) Curr. Opin. Pulm. Med. 9:28-33. The MMPs, which include the collagenases, gelatinases, stromelysin, and macrophage elastase, have similar structures, with an amino terminal domain, a fibronectin-like domain, a zinc-binding domain, and a C-terminal domain. In addition, some members incorporate a transmembrane domain and α2V collagen-like domain. The MMPs are characterized by an active-site zinc atom that is chelated by three histidine residues.

Many MMPs are implicated in the pathogenesis of respiratory diseases, and macrophage elastase (ME, or MMP-12) is an MMP that is implicated in smoking-induced emphysema. It has been shown that mice that are genetically lacking in ME do not develop emphysema when exposed to tobacco smoke. MMP-9 is the predominant MMP in asthma.

The MMPs are directly inhibited by matrix metalloprotease inhibitors (MMPIs). Direct inhibition involves inhibition of the MMP itself, rather than inhibition of a precursor or enzymatic step in the production of the MMP. Natural MMPIs include the tissue inhibitors of matrix metalloproteases, or TIMPs, which are present in all connective tissue. The methods of the invention generally utilize synthetic MMPIs. A “synthetic MMPI,” as used herein, is a non-naturally occurring substance that reduces or eliminates the activity of one or more MMPs. A synthetic MMPI may have any structure or design, and may reduce or eliminate MMP activity through any mechanism. In general such inhibitors are designed to contain a moiety that mimics part of the peptide sequence that is bound by the MMP and a moiety that chelates the zinc atom of the MMP. In some embodiments of the invention, the synthetic MMPI used contains hydroxamate zinc-chelating group. See FIG. 1. In some embodiments, the methods of the invention utilize an MMPI selected from the group consisting of ilomastat, batimastat, marimastat, prinomastat, CGS 27023A, BAY 12-9566, Ro 32-3555, RS-113456, RS-132908, and RS-130,830. A summary of the properties of these MMPIs is given in Table 1. In some embodiments of the invention, the synthetic MMPI is ilomastat or a similar compound, i.e., an MMPI with a hydroxamate zinc-chelating site and binding moieties that include L-trp and isobutyl groups; such compounds useful in the invention are disclosed in Levy et al. (1998) J. Med. Chem. 41:199-223, and Table 1 of U.S. Pat. No. 5,183,900. Preferred MMPIs for use in the methods of the invention are those which have a broad spectrum of inhibition for MMPs, such as those in Table 1 of this application.

TABLE 1
CHELATING MOIETY AND INHIBITORY PROFILE
OF SELECTED MMPIs

| | | | | | | | | MMP-12 |
|---|---|---|---|---|---|---|---|---|
| | Zinc- | MMP-1 | MMP-2 | | | MMP-8 | MMP-9 | (macrophage |
| | chelating | (collagenase- | (gelatinase | MMP-3 | | (collagenase- | (gelatinase | metalloelastase, |
| MMPI | moiety | 1) | A) | (stromelysin) | MMP-7 | 2) | B) | ME) |
| ilomastat | hydroxamate | 0.4 | 0.4-0.5 | 26-27 | — | 0.1-0.2 | 0.2-0.6 | 0.2-0.4 nM |
| batimastat | hydroxamate | — | — | — | — | — | — | — |
| marimastat | hydroxamate | 5* | 6* | 200* | 20* | — | 8* | — |
| prinomastat | hydroxamate | 8 | 0.08 | 0.27 | 54 | — | 0.26 | — |
| CGS 27023A | hydroxamate | 33 | 20 | 43 | — | — | 8 | — |
| Ro 32-3555 | hydroxamate | 3 | 154 | 527 | — | — | 59 | — |
| RS-113456 | hydroxamate | 70 | — | 5.2 | — | — | 0.065 | 0.033 |
| | | (0.089)** | | | | | | |
| RS-132908 | hydroxamate | 360 | — | 12 | — | — | 0.41 | 0.12 |
| | | (0.30)** | | | | | | |
| RS-130,830 | hydroxamate | 590 | 0.22 | 9 | 1200 | — | 0.58 | — |

All values are Kₗ, nM, except *IC₅₀, nM
**values in parentheses are for Membrane-type MMP-1

In some embodiments of the invention, the MMPI used is ilomastat (FIG. 1). Ilomastat is a highly potent broad-spectrum synthetic inhibitor of MMPs that contains a hydroxamate zinc-binding moiety, and that is a modified dipeptide analog with the structure N-[2(R)-2(hydroxyamidocarbonylmethyl)-4-methylpentanoyl]-L-tryptophan methylamide. Ilomastat inhibits a variety of MMPIs, including MMPIs 1, 2, 8, 9, and 12 (collagenases 1 and 2, gelatinases A and B, and macrophage elastase). See Table 1. See also, e.g., Grobelny et al. (1992) Biochemistry 31:7152-4, Levy, et al. (1998) J. Med. Chem. 41:199-223 and Galardy, R. E. (1993) Drugs of the Future 18:1109-1111. Ilomastat is available from, e.g., AMS Scientific Inc. PO Box 273269 Concord Calif., 94527, and is manufactured under the trade name GALARDIN; it is also available from CalBiochem.

Other Active Ingredients

In some embodiments, the synthetic MMPI is administered in combination with one or more other active ingredients. Pharmaceutically active agents useful in the invention include, without limitation, bronchodilators, corticosteroids, and other agents useful in the treatment of a respiratory disease associated with MMPs.

Bronchodilators are medications that increase the forced expiratory volume (FEV) or change other spirometric values, usually by altering airway smooth muscle tone and widening the airways. Bronchodilators useful in the methods of the invention include β2-agonists, anticholinergics, and methylxanthines, or combinations thereof. β2-agonists include fenoterol, salbutamol (albuterol), terbutaline, formoterol, and salmeterol. Anticholinergics include ipratropium bromide, oxitropium bromide, and tiotropium bromide. Ipratropium bromide is available under the tradename ATROVENT (Boehringer Ingelheim). Tiotropium bromide is available under the tradename SPIRIVA (Boehringer Ingelheim and Pfizer Inc.). Methylxanthines include aminophylline and theophylline.

Corticosteroids useful in the methods of the invention include, for example, beclomethasone dipropionate, triamcinolone acetonide, fluticasone propionate, flunoside, and budesonide, and combinations thereof.

Other agents that may be useful in the methods of the invention include mucolytics/expectorants for mucus regulation, and antibiotics for the management of infection, if present.

Dosages and formulations for these agents are well-known in the art. See, e.g., NIH, GOLD report.

Administration and Dosage

The methods of the invention encompass administration of a synthetic MMPI, optionally with other active ingredients, by inhalation, and the formulations of the invention include those that are suitable for storage of the active ingredients, and for administration by inhalation. For a detailed description of formulations useful in the methods of the invention, see Compositions of the Invention, below.

As used herein, “administration by inhalation” or “inhalation” refers to a route of administration that delivers an effective amount of the compound so administered to the tissues of the lower respiratory tract. Such administration entails inhalation of the subject compound by the individual, thereby drawing the compound into the deep lung. In addition, as used herein, “deliver” is synonymous with “administer,” and “delivery” is synonymous with “administration.”

Methods of administration of pharmaceuticals and other substances by inhalation are well-known. In general, the anatomy of the lungs requires that compounds are delivered as aerosols with a particle range of about 0.5 to about 6 μm. Methods known in the art to generate and deliver such aerosols include nebulizers (liquid formulations), dry powder inhalers (dry powder formulations), and metered dose inhalers (drug formulation suspended in a propellant that evaporates virtually instantaneously). Such delivery methods are well-known in the art. See, e.g., M. Keller (1999) Int. J. Pharmaceutics 186:81-90; M. Everard (2001) J. Aerosol Med. 14 (Suppl 1):S-59-S-64; Togger and Brenner (2001) Am. J. Nursing 101:26-32. Commercially available aerosolizers for liquid formulations, including jet nebulizers and ultrasonic nebulizers, are useful in the methods of the invention. For delivery in liquid form, liquid formulations can be directly aerosolized and lyophilized powder can be aerosolized after reconstitution. For delivery in dry powder form, the formulation may be prepared as a lyophilized and milled powder. In additions, formulations may be delivered using a fluorocarbon formulation or other propellant and a metered dose dispenser. For delivery devices and methods, see, e.g., U.S. Pat. Nos. 4,137,914; 4,174,712; 4,524,769; 4,667,688; 5,672,581; 5,709,202; 5,780,014; 5,672,581; 5,915,378; 5,997,848; 6,123,068; 6,123,936; 6,397,838.

For the purpose of the present invention, the appropriate dosage regimen, i.e., dose, timing and repetition, of synthetic MMPI will depend on the MMPI employed, whether the MMPI is administered in combination with other active ingredients, the formulation used (especially whether the formulation is a liquid or dry powder), whether the agent is administered for preventive or therapeutic purposes, the type and severity of the respiratory disease associated with MMPs to be treated or prevented, previous therapy, the patient's clinical history and response to the agent, genetic factors such as known AAT deficiency, and the discretion of the attending physician, if the individual is under the care of a physician.

A single dose or repeated doses may be given of one or more agents described herein. For repeated administrations over several days or longer, depending on the condition, the treatment is sustained until a desired suppression of disease symptoms occurs or until sufficient levels of MMPI and other active ingredients, if used, are achieved to reduce the risk of a respiratory disease associated with MMPs under the environmental conditions to which the treated individual is exposed. In some embodiments, the dosage regimen is designed to reduce the risk of emphysema in a smoker. In the case of treatment of established respiratory disease associated with MMPs, the progress of therapy is easily monitored by conventional techniques and assays. In the case of prevention of respiratory disease associated with MMPs, e.g., in smokers, the treatment may continue indefinitely, e.g., as long as exposure to tobacco smoke lasts. For treatment for hereditary conditions, such as hereditary emphysema, treatment may continue throughout the lifetime of the individual. The dosing regimen can vary over time, and may be adjusted according to disease progression or remission, or exposure to environmental factors that cause or aggravate respiratory disease associated with MMPs, or a combination of these.

A striking property of the methods of the present invention is the dosage required to completely prevent the appearance of indices of respiratory disease associated with MMPs in an individual who is exposed to tobacco smoke. Typically, oral dosages of MMPIs required to be effective in treating or preventing COPD are about 50 to 100 mg/kg/day, the equivalent of an oral dose of about 3.5 to 7 gm/day in a 70 kg human. In terms of pulmonary delivery of antiproteases to treat or prevent COPD, typically, dosages of antiproteases, e.g., AAT, required to be given by pulmonary delivery to treat environmental or genetic predisposition to COPD are in the range of hundreds of milligrams per day. For example, dosage levels of AAT used in previous and/or current clinical studies are about 50-200 mg/day of liquid aerosol preparations. In contrast, in some embodiments the methods of the invention require a cumulative daily dosage level (delivered as a single dose or as divided doses) of less than 100 mg/day, in some embodiments, less than 10 mg/day, in further embodiments, less than 1 mg/day, and in yet further embodiments, less than 0.3 mg/day (in some embodiments, less than about 100 mg/day, about 10 mg/day, about 1 mg/day, or about 0.3 mg/day) of ilomastat, in liquid solution or as a dry powder, to treat or to prevent (e.g., eliminate the appearance, increase the time to appearance, delay or slow the development, and/or decrease the number and severity of clinical and other manifestations) a respiratory disease associated with MMPs (for example, COPD). In one embodiment, the invention provides methods of treatment and/or prevention of emphysema (in some embodiments, in a smoker) by inhalation of ilomastat where the daily dose (in some embodiments, as a single dose and in some embodiments as divided doses) of ilomastat is less than about 10 mg/day. In another embodiment, the invention provides methods of treatment and/or prevention of asthma by inhalation of ilomastat where the daily dose (in some embodiments, as a single dose and in some embodiments as divided doses) of ilomastat is less than about 0.3 mg/day.

Typically the individual will administer an MMPI, such as ilomastat, until a dosage is reached that achieves the desired result. As will be appreciated by the skilled artisan, the size of a single dose of an MMPI, e.g., ilomastat, to be delivered by pulmonary administration depends on the form of the MMPI used (i.e., liquid or dry powder), the likely volume to be inhaled, and, in the case of a liquid, the solubility of the MMPI. In addition, MMPIs vary in their inhibition profiles for the various MMPs (see Table 1), and dosage will be adjusted based on the inhibitory profile of a given MMPI. Inhibition constants for representative MMPIs are given in Table 1. Further inhibition constants may be found in the literature, e.g., Levy, et al. (1998) J. Med. Chem. 41:199-223, and U.S. Pat. No. 5,183,900. In addition, methods of determining inhibition constants for an MMPI with individual MMPs are well-known in the art and routinely used by those of skill in the art. Doses (in some embodiments, as a single dose and in some embodiments as divided doses) contemplated in the methods of the invention range from a lower limit of about any of 0.01 mg, 0.05 mg, 0.1 mg, 0.5 mg, 1 mg, 2 mg, 3 mg, 4 mg, 6 mg, 10 mg, 20 mg, or 50 mg to an upper limit of about any of 0.1 mg, 0.5 mg, 1 mg, 2 mg, 4 mg, 6 mg, 8 mg, 12 mg, 20 mg, 50 mg, or 100 mg. In some embodiments, the daily dose (in some embodiments, as a single dose and in some embodiments as divided doses) is less than about 80, or 40, or 20, or 10, or 5, or 1, or 0.5 mg/day. As an example, ilomastat is soluble in liquid formulation at levels up to 15 mg/ml, depending on the formulation, and a typical volume of one inhalation is 0.05 mL to 4 mL. Thus, a single dose of ilomastat taken in liquid form via a nebulizer could contain a dose of ilomastat up to 60 mg, as required by the severity of the disease, the environmental and hereditary factors affecting the individual treated, and other factors apparent to the attending physician. Generally, when used for, e.g., prevention of emphysema in a smoker with no other risk factors for emphysema, a single dose (e.g., daily, or as one of two or more doses during the day) of ilomastat will be in the range of about 0.1 mg to about 20 mg, or about 0.1 mg to about 12 mg, or about 1 mg to about 10 mg, or about 4 mg to about 8 mg, or about 6 mg. The administration may be self-administration, and the ilomastat may be in liquid or dry powder formulation. An individual, such as a smoker, may, for example, administer the ilomastat formulation in the morning, or periodically throughout the day, or before each exposure to tobacco smoke. It will be readily apparent to one of skill in the art that the daily dose size may be adjusted to account for the frequency and timing of administration of the ilomastat, and that the daily dosage may, to some degree, be determined by the individual or a clinician based on estimated exposure to tobacco smoke and the type of exposure (e.g., passive or active), on the delivery system used (e.g., dosage required in a dry powder formulation can be different, e.g., lower, than those in a liquid nebulizer; dosage in a metered dose inhaler may also require adjustment), and on the presence or absence of other risk factors (e.g., hereditary risk factors for emphysema, or other environmental risk factors such as occupational risk factors and/or exposure to air pollution). In addition, it may be desirable to place an upper limit on single doses and/or daily dosage. Administration devices that limit or modulate self-administration of pulmonarily-administered pharmaceuticals and other substances in order to prevent possible overdose by the individual are well-known in the art.

Dose frequency (i.e., frequency of administration) may be from once daily, twice daily, or three times daily, to twice daily, four times daily, six times daily, eight times daily, or more than eight times daily. In some embodiments, the dose frequency is from once daily to six times daily, or once daily to four times daily, or once or twice daily. In some embodiments the frequency of administration is twice daily. Frequency of administration may be determined and adjusted over the course of treatment or prevention, and is generally, but not necessarily, based on treatment and/or suppression and/or amelioration and/or delay of symptoms and clinical findings. In the case of prevention of a respiratory disease related to matrix metalloproteases, in an individual exposed to environmental conditions that increase the likelihood of a respiratory disease related to matrix metalloproteases, frequency of administration may be modulated based on the frequency and/or severity of exposure. In one embodiment of the invention, smokers self-administer the formulations of the invention at a frequency that depends on the frequency of cigarette smoking or exposure to secondhand smoke. An exemplary dosage frequency and size for ilomastat, in a smoker exposed to 10 to 40 (e.g., 20) cigarettes per day with no other risk factors for emphysema, is contemplated as about 6 mg, in one dose, once per day. Higher or lower doses may be used. In some embodiments, a once-daily dosage is about 0.5, 1, 2, 3, 4, 5 or 6 mg. In some embodiments, a daily cumulative dosage, given as two divided doses, is about 0.5, 1, 2, 3, 4, 5 or 6 mg. Greater or lesser frequency of administration may also be used. The size and frequency of dosage may be determined at the discretion of a clinician, depending on factors previously discussed.

As will be appreciated by one of skill in the art, individual respiratory diseases associated with MMPs are likely to have unique profiles for MMPs involved in the disease, and for the typical elevation of levels of MMPs. Hence dosage size and frequency may be adjusted according to the disease state treated. For example, treatment or prevention of asthma may require a daily dose (in some embodiments, as a single dose and in some embodiments as divided doses) of an MMPI from about 0.010 mg to about 1 mg per day, or from about 0.02 mg to about 0.5 mg per day, or from about 0.05 mg to about 0.3 mg per day. In one embodiment of the invention, the MMPI administered at these doses is ilomastat. The size and frequency of dosage may be determined at the discretion of a clinician, depending on factors previously discussed.

In some embodiments, the amount of MMPI administered by inhalation is less than about ½, ¼, 1/10, 1/50, or 1/100 of the dose of the same MMPI used to treat the same MMP-related respiratory disease (e.g., COPD, such as emphysema) when administered by a non-inhalation route.

When other active ingredients are administered in “combination” or in “conjunction” with the MMPI, they may be administered simultaneously, in the same or different formulations, at separate times, on the same or separate schedules, or any combination of the preceding. The dose, frequency, and duration for each agent given above may be combined in any combination to produce a therapeutic effect. In some embodiments, a MMPI is administered in combination with a bronchodilator. In some embodiments, administration frequency of the MMPI in combination with a bronchodilator is once daily. In some embodiments, administration frequency of the MMPI in combination with a bronchodilator is twice daily. In some embodiment, the MMPI is ilomastat. In some embodiments, the MMPI is ilomastat and the bronchodilator is ipratropium bromide, oxitropium bromide, or tiotropium bromide. In some embodiments, the MMPI is ilomastat and the bronchodilator is tiotropium bromide. Dose size and frequency for the MMPI, e.g., ilomastat, are as discussed above. Dose size and frequency for the bronchodilator are well-known in the art. It will be appreciated by those of skill in the art that the combination of the two agents may result in a reduction of dose size or frequency for one or both of the agents compared to either agent administered singly, in order to achieve a therapeutic effect.

Exemplary methods of the invention include, but are not limited to: treatment of an individual who suffers from emphysema by administration of ilomastat in a cumulative daily dose of about 1 mg to about 10 mg by inhalation; prevention of emphysema in an individual susceptible to emphysema, e.g., a smoker (especially, an active smoker), by administration of ilomastat in a cumulative daily dose of about 1 mg to about 10 mg by inhalation; treatment of asthma in an individual who suffers from asthma by administration of ilomastat in a cumulative daily dose of about 0.1 mg to about 1 mg by inhalation. All of the above embodiments may also include the administration of another active agent in combination with the ilomastat, for example, oxitropium bromide, ipratropium bromide, or tiotropium bromide.

Indices of Efficacy

Treatment efficacy can be assessed by methods well-known in the art. Indices of efficacy include, but are not limited to, reduction of, or halting or slowing of progression of, clinical manifestations of disease such as dyspnea, coughing, wheezing (especially on forced exhalation), reduction in forced expiratory volume (FEV), reduced arterial PO₂, dependent edema, and recurrent respiratory infection. Indices of efficacy of prevention of disease, e.g., in an individual exposed to environmental conditions that increase the likelihood of COPD, e.g., a smoker, include failure of COPD to appear, or a delay in the time to appearance of COPD compared to the expected time to appearance for a similar, untreated individual, or a slowing or halting of COPD if present or if it does appear. The COPD may be emphysema. Quality of life measures may also be used to assess efficacy, such as physical functioning, bodily pain, general health, vitality, social functioning, decreasing the dose of other medications, e.g. palliative care medications or other medications, required to treat the disease, delaying the progression of the disease, decreasing time required for resolution of secondary infection and/or symptoms, and/or prolonging survival of patients. Other clinical indices are known to those of skill in the art.

Compositions of the Invention

The invention also encompasses compositions useful to treat or prevent a respiratory disease associated with MMPs by inhalation. The compositions of the invention contain at least one MMPI. In one embodiment, the MMPI is ilomastat.

Compositions of the invention may be in liquid form, and contain the MMPI and other optional active ingredients in an aqueous carrier or other suitable carrier for use in pulmonary administration. Preparation of liquid compositions may be carried out using a variety of well-known methods, such as are described in Remington, The Science and Practice of Pharmacy 20th Ed. Mack Publishing (2000). The MMPI may be in a solution or in suspension, or may be prepared as a lyophilized composition that is reconstituted prior to administration. The liquid compositions may contain other active ingredients, such as bronchodilators and/or corticosteroids, as well as suitable pharmaceutically acceptable excipients. Acceptable carriers, excipients, preservatives and/or stabilizers are nontoxic to recipients at the dosages and concentrations employed, and are well-known to those of skill in the art. Preservatives are optionally included in the composition used in the invention to maintain the integrity of the composition. The compositions to be used for in vivo administration are preferably sterile. This is readily accomplished by, for example, filtration through sterile filtration membranes. See, e.g., Example 1.

Dry powder compositions may also be used. Preparation of dry powder compositions may be carried out using a variety of well-known methods, including lyophilization, spray drying, agglomeration, spray coating, extrusion processes, and combinations of these. Methods are aimed at producing a composition that is a substantially amorphous powder of homogenous constitution having a particle size that is readily respirable, has a low moisture content, and has flow characteristics that allow for ready aerosolization. In some embodiments it may be useful to prepare the powder as an aggregate or agglomerate composition in order to improve handling characteristics such as flowability, low caking and the like. These dry powder compositions comprise an MMPI, e.g., ilomastat, in a therapeutically or prophylactically effective amount together with one or more pharmaceutically or therapeutically acceptable carriers and optionally other active ingredients. Such ingredients, and considerations involved in the preparation of compositions for dry powder delivery, are described in, e.g., Gillman et al. (eds.) (1990) Goodman and Gilman's: The Pharmacological Bases of Therapeutics, 8^{th} Ed., Pergaman Press; Norris (ed.) (1989) Novel Drug Delivery Systems, 2^{nd} Ed., Marcell Dekker Inc.; Remington 's Pharmaceutical Sciences 19^{th} ed. (2000) Mack Publishing Co.; and U.S. Pat. No. 5,780,914 and references therein.

The composition may be provided in any suitable container known in the art or apparent to the ordinarily skilled artisan. Compositions may be prepared in unit dosage form, e.g., at the dosages given above, or in multiples of those dosages, for use in nebulizers, as is common in the art. See, e.g., U.S. Pat. Nos. 4,137,914; 4,174,712; 4,524,769; 4,667,688, 5,780,014, 5,672,581; 5,915,378; and 5,997,848.

Kits of the Invention

The invention further encompasses kits for use in the methods described above. Kits may include the compositions of the invention, in suitable containers, and any materials necessary or useful in the administration and use of the compositions in the methods described above. In some embodiments, the matrix metalloprotease inhibitor is formulated for inhalation. In some embodiments, the matrix metalloprotease inhibitor is ilomastat. In some embodiments, the kit contains a device to aerosolize the composition(s). In some embodiments of the invention the composition(s) is/are provided in a container, and optionally further packaging for segregation from other components of the kit and/or to facilitate dispensing, and a set of instructions for use of the composition(s). The instructions may inform the user of methods for administration of the composition(s) of the invention, suggested dosages and schedules for various levels of exposure to environmental conditions that promote COPD (e.g., emphysema), such as smoking or air pollution, precautions, expected results, warnings concerning improper use, and the like. The instructions may be in any form, and provided, e.g., as a separate insert or on a label that is affixed to the container or packaging. Instructions include instructions for any of the methods described herein. In some embodiments, instructions are directed to the use of ilomastat by inhalation in the prevention or treatment of emphysema. In some embodiments, instructions are directed to the use of ilomastat by inhalation in the prevention or treatment of emphysema in a smoker. In some embodiments, instructions are directed to the use of an MMPI by inhalation in the prevention or treatment of hereditary emphysema. In some embodiments, the MMPI is ilomastat, and the instructions are directed to the use of ilomastat by inhalation in the prevention or treatment of hereditary emphysema. In some embodiments, instructions are to the use of an MMPI by inhalation in the prevention or treatment of asthma. In some embodiments, instructions are to the use of ilomastat by inhalation in the prevention or treatment of asthma.

Exemplary optional additional components of kits of the invention include diluents for compositions to be reconstituted, and components to facilitate use of the aerosolizer.

The present invention is further illustrated by the following examples.

### EXAMPLES

### Example 1

This example demonstrates the preparation of a synthetic MMPI, ilomastat, in a solution suitable for pulmonary administration via a nebulizer.

Materials required:
Phosphate buffered saline (PBS), pH 7.4
Lyophilized ilomastat (>95% pure on its product specification)
0.45 um Acrodisc filter (Product #4654 from GelmanSciences)

Procedures:
1. ˜3 mg of ilomastat was weighed out.
2. The ilomastat was dissolved in a 10 mL volume of 1×PBS, pH 7.4
3. The mixture was vortexed for a minimum of 2 minutes.
4. The mixture was let sit at room temperature (RT) overnight.
5. The mixture was vortexed again before filtering the solution to remove undissolved ilomastat.
6. The solution was filtered using a 0.45 um Acrodisc filter.
7. The absorbance of the filtered solution was measured at 280 nm.
8. Peptide concentration was determined by dividing the measured absorbance by the extinction coefficient for ilomastat of 14.16. The solution was used if the final concentration was in the range of 85-110 ug/mL.

### Example 2

This example shows the efficacy of ilomastat administered by pulmonary delivery in the prevention of indices of emphysema in an established model, the murine model of cigarette smoke-related emphysema.

As is known in the art, mice tolerate at least two cigarettes daily for many months, resulting in pulmonary changes similar to human emphysema. Hence this is a widely-used model for smoking-induced emphysema that correlates to results seen in humans. See, e.g., Shapiro (2000) Am. J. Respir. Cell Mol. Biol. 22: 4-7; Hautamki et al. (1997) Science 277: 2000-2004.

Treatment Groups:

Five groups of animals were studied. (See Table 2). Group 1 animals (32 animals) were control non-smoking animals. Group 2 animals (32 animals) were administered aerosolized PBS (10 ml/5 animals) followed by cigarette smoke. Group 3 animals (32 animals) received high-dose aerosolized ilomastat (100 ug/ml in PBS; 10 ml/5 animals) and then smoked. Group 4 animals (32 animals) received mid-dose aerosolized ilomastat (50 ug/ml in PBS; 10 ml/5 animals) and then smoked. Group 5 animals (32 animals) were administered low-dose aerosolized ilomastat (10 ug/ml in PBS; 10 ml/5 animals) for 30 minutes and then smoked. All adult mice (12 weeks of age at initiation) were exposed to two cigarettes per day, 6 days/wk for up to 6 months.

TABLE 2
TREATMENT GROUPS FOR MOUSE EXPOSURE
TO CIGARETTE SMOKE AND ILOMASTAT

| | | Total daily | Calculated | Calculated |
|---|---|---|---|---|
| | | dose of | dose | dose |
| | | ilomastat, | delivered to | delivered |
| Group | | per | breathing | to |
| # | Treatment | mouse (μg) | zone (μg) | lungs (μg) |
| 1 | Non Smokers | — | — | — |
| 2 | Sm (Vehicle only) | 0 | — | — |
| 3 | Sm (ilomastat dose 1) | 200 | 8 | 0.4 |
| 4 | Sm (ilomastat dose 2) | 100 | 4 | 0.2 |
| 5 | Sm (ilomastat dose 3) | 20 | 0.8 | 0.04 |

Sm—Smoke

Within each treatment group there were 3 timepoints for analysis: 1 week, 3 months, and 6 months. After one week, 3 months, and 6 months on study, 10 animals, 10 animals, and 12 animals respectively, from each group were sacrificed and analytical work on lungs and lung samples was performed (see below).

The animals (five at a time) were placed in the drug delivery device, which delivered five separate, equal doses of ilomastat, one to each mouse, as an aerosolized liquid. 10 mL of sterile saline or drug was placed in the nebulizer and delivered to each group of five animals. Delivery proceeded until the nebulizer ran dry (about 30 minutes).

Three concentrations of ilomastat were used: 100 ug/mL (ilomastat dose 1), 50 ug/ml (ilomastat dose 2) and 10 ug/ml (ilomastat dose 3). All were prepared in sterile saline. Vehicle control animals received sterile saline only.

Analyses:

In each animal, the right lung was removed and used for fixation/inflation and paraffin embedding to study morphometry, to determine mean linear intercept (Lₘ), which is the distance between the walls of alveoli, and which increases as proteases (e.g., elastase, metalloproteases) break down pulmonary structure.

The left lung was subjected to bronchoalveolar lavage (BAL) to determine levels of the inflammatory cell types macrophages and neutrophils, which were counted by morphology. BAL samples were collected using 0.5 mL rinses of the left lung.

Results

### Lm Data

| | Lm, μm | vs. non- | | P vs. smokers/ |
|---|---|---|---|---|
| Group | (S.D.) | smokers (%) | % protection | non-smokers |
| non-smokers | 34.4 (3.4) | — | — | 0.0006 |
| smokers | 39.4 (1.4) | 15 | — | 0.0006 |
| ilomastat, | 35.3 (1.7) | 2.6 | 83 | 0.003/0.16 |
| dose 1 | | | | |
| ilomastat, | 34.6 (1.8) | 0.6 | 96 | <0.0001/0.61 |
| dose 2 | | | | |
| ilomastat, | 37.4 (3.5) | 8.7 | 42 | 0.001/0.024 |
| dose 3 | | | | |

### Inflammatory Cell Data

| | Neutrophils, cells per ml | Macrophages, cells per |
|---|---|---|
| Group | of BAL fluid | ml of BAL fluid |
| non-smokers | 80 | 7500 |
| smokers | 470 | 27,000 |
| ilomastat, dose 1 | 100 | 1000 |
| ilomastat, dose 2 | 220 | 1500 |
| ilomastat, dose 3 | 50 | 2500 |

This Example demonstrates significant reduction of indices of COPD (emphysema in this case) by inhalation of an MMPI, ilomastat, in animals chronically exposed to cigarette smoke, an established cause of COPD. Strikingly, a very low dose of ilomastat was very effective, by the measures of this study.

### Example 3

Two animal studies of 1 week or 2 weeks duration were carried out to determine if Ilomastat, delivered by nebulization, could inhibit MMP activity present in BAL samples obtained from smoking mice. In the 1 week study 5 animals/group were treated with either ilomastat (100 ug/mL) or vehicle buffer (PBS) daily and then exposed to smoke as described in the protocol for the 6 month smoking mouse study (Example 2). The 2-week study was a repeat of the 1-week study but used 10 animals/group.

At the end of each study, BAL samples were obtained from the lungs of each animal using established procedures and each sample was tested for MMP activity using an assay designed primarily to detect MMP-9 activity.

A. Assay of MMP Activity

1. 150 μL of MMP Reagent Mix was added to each well. The reagent mix consisted of the appropriate dilutions of previously prepared stock solutions in order to yield the following final concentrations in 200 μL final volume/well:
   HEPES stock (50 mM final)
   CaCl₂ stock (10 mM final)
   Ellmans Reagent stock (1.25 mM final)
   Substrate (Bachem #H7145) stock (475 μM final)
   Q.S. with dH₂O
2. Microtiter plate was placed in the 30° C. incubator for 15 min.
3. 50 μL/well of each BAL sample was added into the appropriate wells
4. 50 μL/well of vehicle control buffer was added to blank well.
5. 48 μL/well of vehicle control buffer and 2 μL of MMP-9 (20 ng, CalBiochem #444231) was added to the control well.
6. Plate was placed in VersaMax Microtiter Plate Reader which was pre-equilibrated to 30° C.
7. Abs₄₁₀ was read every 5-6 min for 3 hrs
   Results and Conclusions:

### MMP Activity of BAL Samples+/−Ilomastat

| Treatment | Sample | Activity (mAU/min) |
|---|---|---|
| Pilot study (1 week) | D1 | 0 |
| Drug treated | D2 | 0 |
| | D3 | 0 |
| | D4 | 0 |
| | D5 | 0 |
| Pilot study (1 week) | C1 | 1.365 |
| Vehicle treated | C2 | 0 |
| | C3 | 0.651 |
| | C4 | 0 |
| | C5 | 0 |
| 2 week study | D947 | 0 |
| Drug treated | D948 | 0 |
| | D949 | 0 |
| | D950 | 0 |
| | D951 | 0 |
| | D1376 | 0 |
| | D1377 | 0 |
| | D1378 | 0 |
| | D1379 | 0 |
| | D1380 | 0 |
| 2 week study | Ve977 | 0 |
| Vehicle treated | Ve978 | 0 |
| | Ve979 | 1.811 |
| | Ve980 | 0 |
| | Ve981 | 0 |
| | Ve1401 | 1.626 |
| | Ve1402 | 0.271 |
| | Ve1403 | 0 |
| | Ve1404 | 0 |
| | Ve1405 | 0 |

The data from the 1-week study indicate that two of the five vehicle-treated mouse samples exhibit detectable levels of MMP activity. All five of the Ilomastat-treated mouse samples possessed no detectable MMP activity. In the 2-week study, three out of ten vehicle-treated mouse samples also exhibited detectable levels of MMP activity, while all ten of the Ilomastat-treated mouse samples possessed no detectable MMP activity. Thus, in each study, MMP activity was observed in 30-40% of the control mice, while all of the Ilomastat-treated mice demonstrated an absence of measurable MMP proteolytic activity.

The results of this Example, taken together with those of Example 2, demonstrate: 1) Inhalation of ilomastat at low doses prior to smoking reduced smoking-induced MMP levels to below detectable limits; 2) Inhalation of ilomastat at low doses prior to smoking reduced smoking-induced inflammatory cell infiltrates to levels comparable with those of non-smoking mice; 3) Inhalation of ilomastat at low doses prior to smoking reduced indices of loss of lung integrity to levels that were not statistically different from those of non-smoking mice.

### Example 4

This Example demonstrates a calculation of a daily dose (in some embodiments, delivered as a single dose and in some embodiments delivered as divided doses) of ilomastat for prevention of smoking-induced emphysema in a human (e.g., a smoker).

1. Mouse Data and Calculations

In the smoking mouse study (Example 2), it was known that: 1) 3 doses of ilomastat were aerosolized: 10 ug/mL, 50 ug/mL, and 100 ug/mL; 2) 10 mL of each dose/day was aerosolized at a time and delivered to 5 mice. Therefore, the theoretical maximum daily dose each mouse could receive is 2 mL of each drug dosage or 20, 100, or 200 ug of ilomastat (The spatial uniformity of delivery is no worse than 13% CV); 3) Maximal efficacy/dose was achieved with the 50 ug/mL dose; and 4) The delivery device delivered approx. 3.8-8.5% (the midpoint of 6.15% was used in these calculations) of aerosolized drug to “the breathing zone” of each animal when nebulizing 1-2 mL volumes of 100 ug/mL ilomastat in sterile PBS.

Two calculations given below were performed to determine deposition in the mouse lung using the mid (50 ug/mL) dose level. The first calculation (Theoretical Maximum Deposition) is based on complete delivery of the theoretical maximum amount of drug (100 ug) to each animals breathing zone. The second calculation (Realistic Practical Deposition) is based on the more realistic practical amount of drug delivered to the breathing zone of each animal (6.15% of 100 ug or, 6.15 ug). In addition, for the first calculation the following assumptions were made: I) 90% nebulizer efficiency; 2) 65% of particles in respirable range; 3) 3-7% deposition in the deep lung (will use 5%). For the second calculation only the second and third assumptions apply.

Calculation 1: Theoretical Maximum Deposition.
100 ug/animals breathing zone
90 ug nebulized
59 ug respirable
3 ug deposited in the lungs of each animal

Calculation 2: Realistic Practical Deposition
6.15 ug/animals breathing zone
4 ug respirable
0.2 ug deposited in the lungs of each animal
   2. Human Dosage Calculation

For calculation of human dosage, the following assumptions were made: 1) mouse weighs 30 g; 2) human weighs 70 kg (×2,000 approx); 3) 25-40% deposition in the lung (33% used in considering the nebulizer charge); 4) Total deposition in humans is 5× less than in mice, due to differences in lung anatomy and breathing rates, i.e. L/min/kg. (Deposition is 5-10× less in rats, 3× less in dogs for 1 um particle sizes. The mouse value of 5× is an extrapolation from the rat).

Two calculations were performed. The first calculation represents the dosage necessary in a human if one wishes to achieve the Theoretical Maximal Deposition that was achieved in the mouse. The second calculation represents the dosage necessary in a human if one wishes to achieve the Realistic Practical Deposition that was achieved in the mouse; i.e., the dosage that was most probably actually achieved in the mouse model (which was much lower than the Theoretical Maximum).

Calculation 1. Theoretical Maximum Deposition calculated for human based on mice data and assumptions:
Body weight adjustment: 3 ug×2,000=6 mg
Lung deposition adjustment: 6 mg×1/0.33=18 mg
Total deposition adjustment: 18 mg×5=90 mg Ilomastat as a single nebulizer charge

Calculation 2. Realistic Practical Deposition calculated for human based on mice data and assumptions:
0.2/3×90=6 mg Ilomastat as a single nebulizer charge to achieve the same deposition as in the smoking mouse study.

This example demonstrates that the dosage of ilomastat required for prevention of emphysema in human smokers is easily calculated. As will be readily apparent to the skilled artisan, dosage size can be adjusted based on adjustments to assumptions and further information, available through routine experimentation.

### Example 5

In this Example a sheep model for asthma was used to test the effect of three different doses of ilomastat on asthma.
3 solutions were used:
1. Phosphate buffered saline (PBS) (control)
2. 30 ug ilomastat in 3 mL PBS
3. 100 ug ilomastat in 3 mL PBS
   These doses were delivered into the lungs of sheep by nebulization and then the lungs were challenged with the bronchoconstrictive agent carbochol either 30 mins later (P-drug) or 24 hours later.
   Results:

DRC/PC %400 refers to the amount of carbochol required to induce a 400% change in airway hyperresponsiveness. The amount required is measured as carbachol breath units (BU); the aerosol concentration of carbachol is fixed and the animal continues to inhale until the appropriate response in obtained. Thus, a higher value indicates a protective effect against an asthmatic reaction. This value (Carbachol BU) is higher in animals treated with ilomastat, in particular the 30 ug dose, indicating that ilomastat at low doses has a prophylactic effect against acute asthmatic responses. Strikingly, the lower dose of only 30 μg had a clear protective effect in this model 24 hours after administration.

| | Carbachol DRC'S/PC 400%-BU | | |
|---|---|---|---|
| | Baseline | 30 min post- | 24 hr post- |
| Test | (S.E.) | ilomastat (S.E.) | ilomastat (S.E.) |
| 100 μg ilomastat | 22.75 (4.52) | 28.75 (1.93) | 31.25 (1.49) |
| 30 μg ilomastat | 26.25 (3.66) | 29.50 (4.37) | 51.25 (11.14) |
| 30 μg ilomastat, repeat | 26.25 (4.19) | 28.00 (4.95) | 35.75 (12.01) |

### Example 6

This example pertains to the inhibition of one kind of MMP (MMP-9) present in induced sputum samples collected from healthy smokers (HS) vs COPD patients.

Methods:

Induced sputum (IS) samples were collected from 22 healthy smokers (HS) and 19 patients with clinical evidence of chronic obstructive pulmonary disease (COPD) (Inclusion criteria: FER<0.7 and FEV₁ % Predicted 30-80%). The IS was processed using dithiothreitol as previously described by Pavord et al. (1997) Thorax 52, 498-501. 15 randomly selected IS were used for inhibitor studies (COPD n=7; HS n=8).

MMP-9 activity in IS was measured using a commercially available human MMP-9 fluorescent activity assay (Fluorokine® E, R&D Systems, Europe, UK). Following binding of MMP-9 (92, 82, 62 kDa forms) to an immobilized monoclonal antibody, a fluorogenic substrate linked to a quencher molecule was added. Any active MMP-9 cleaved the peptide linkers between the fluorophore and the quencher molecule allowing measurement of the fluorescence using a fluorimeter (Spectrafluor Plus, Tecan, UK). For inhibitor studies, the IS samples were incubated for 30 minutes at room temperature with the inhibitors prior to addition of the synthetic substrate.

Ilomastat (30 uM) had a slight but non-significant (ns) inhibitory effect on MMP-9 activity in IS from COPD, 9.2+/−7.2%; p=0.249. Ilomastat (30 uM) significantly inhibited MMP-9 activity in IS from HS, 22.6+/−6.7%; p=0.015. Ilomastat 100 uM had a significant inhibitory effect of IS from both COPD, 42.5+/−9.3%; p=0.004 and HS, 39.4+/−6.3%; p=0.008.

This Example demonstrates that healthy smokers as well as individuals with COPD have MMP (MMP-9) in lung samples (i.e., induced sputum), and that ilomastat can significantly inhibit the MMP-9.

The foregoing written specification is considered to be sufficient to enable one skilled in the art to practice the invention. It is understood that the examples and embodiments described herein are for illustrative purposes only and that various modifications or changes in light thereof will be suggested to persons skilled in the art and are to be included within the spirit and purview of this application and scope of the appended claims.

All publications, patents and patent applications cited herein are hereby incorporated by reference in their entirety for all purposes to the same extent as if each individual publication, patent or patent application were specifically and individually indicated to be so incorporated by reference.

1. A method of treating an individual who suffers from a respiratory disease associated with matrix metalloproteases by administering to the individual an effective amount of a synthetic matrix metalloprotease inhibitor by inhalation, wherein the matrix metalloprotease inhibitor is selected from the group consisting of ilomastat, RS-113456, and RS-132908.

2. The method of claim 1 wherein the matrix metalloprotease inhibitor is ilomastat.

3. The method of claim 2 wherein the ilomastat is administered in a daily dose from about 0.1 to about 20 mg.

4. The method of claim 1 wherein the matrix metalloprotease inhibitor is administered in a cumulative daily dose of about 0.1 mg to about 20 mg.

5. The method of claim 1 wherein the respiratory disease is selected from the group consisting of chronic obstructive pulmonary disease, emphysema, asthma, chronic bronchitis, and cystic fibrosis.

6. The method of claim 1 wherein the respiratory disease is emphysema.

7. The method of claim 6 wherein the individual is a mammal.

8. The method of claim 6 wherein the individual is a human.

9. The method of claim 6 wherein the individual is a smoker.

10. The method of claim 6 wherein the matrix metalloprotease inhibitor is administered in a cumulative daily does of about 1 mg to about 10 mg.

11. The method of claim 10 wherein the matrix metalloprotease is ilomastat.

12. The method of claim 11 further comprising administering another active agent in combination with the ilomastat, wherein the other active agent is selected from the group consisting of oxitropium bromide, ipratropium bromide, and tiotropium bromide.

13. The method of claim 1 wherein the respiratory disease is asthma.

14. The method of claim 13 wherein the matrix metalloprotease inhibitor is administered in a cumulative daily dose of about 0.01 mg to about 1.0 mg.

15. The method of claim 14 wherein the matrix metalloprotease inhibitor is ilomastat.

16. The method of claim 15 further comprising administering another active agent in combination with the ilomastat, wherein the other active agent is selected from the group consisting of oxitropium bromide, ipratropium bromide, and tiotropium bromide.

17. The method of claim 1 further comprising administering another active agent in combination with the matrix metalloprotease inhibitor.

18. The method of claim 17 wherein the other active agent is selected from the group consisting of a bronchodilator and a corticosteroid.

19. The method of claim 18 wherein the other active agent is a bronchodilator and the bronchodilator is selected from the group consisting of oxitropium bromide, ipratropium bromide, and tiotropium bromide.

20. A method of treating an individual who suffers from a respiratory disease associated with matrix metalloproteases by administering to the individual an effective amount of a synthetic matrix metalloprotease inhibitor by inhalation, wherein the matrix metalloprotease inhibitor is administered in a cumulative daily dose of about 0.01 mg to about 20 mg.

21. The method of claim 20 wherein the respiratory disease is chronic obstructive pulmonary disease.

22. The method of claim 21 wherein the respiratory disease is emphysema.

23. The method of claim 22 wherein the individual is a smoker.

24. The method of claim 20 wherein the respiratory disease is asthma.

25. The method of claim 20 further comprising administering another active agent in combination with the matrix metalloprotease inhibitor.

26. The method of claim 25 wherein the other active agent is selected from the group consisting of a bronchodilator and a corticosteroid.

27. A method of preventing a respiratory disease associated with matrix metalloproteases in an individual who is susceptible to or who suffers from a respiratory disease associated with matrix metalloproteases by administering to the individual an effective amount of a synthetic matrix metalloprotease inhibitor by inhalation.

28. The method of claim 27 wherein the matrix metalloprotease inhibitor is selected from the group consisting of ilomastat, RS-113456, and RS-132908.

29. The method of claim 28 wherein the matrix metalloprotease inhibitor is administered in a cumulative daily dose of about 1 mg to about 10 mg.

30. The method of claim 28 wherein the matrix metalloprotease inhibitor is ilomastat.

31. The method of claim 29 wherein the respiratory disease is chronic obstructive pulmonary disease.

32. The method of claim 31 wherein the respiratory disease is emphysema.

33. The method of claim 32 wherein the individual is a smoker.

34. The method of claims 33 further comprising administering another active agent, wherein the other active agent is selected from the group consisting of oxitropium bromide, ipratropium bromide, and tiotropium bromide.

35. A method of preventing emphysema in a smoker by administering to the smoker a cumulative daily dose of ilomastat of about 1 mg to about 10 mg by inhalation.

36. A kit for use in the treatment of a respiratory disease associated with matrix metalloproteases comprising a composition comprising a synthetic matrix metalloprotease inhibitor and instructions for its use.

37. The kit of claim 36 wherein the matrix metalloprotease inhibitor is ilomastat.
